(19) United States
(12) Patent Application Publication
(10) Pub. No.: US 2024/0050067 A1
(43) Pub. Date: Feb. 15, 2024

(54) ULTRASONIC PROBE AND ULTRASONIC DIAGNOSTIC APPARATUS

(71) Applicant: CANON MEDICAL SYSTEMS CORPORATION, Otawara-shi (JP)

(72) Inventors: Shohei SASAKI, Otawara (JP); Yasushi KAMEWADA, Otawara (JP); Sakie TAKEDA, Nasushiobara (JP)

(73) Assignee: CANON MEDICAL SYSTEMS CORPORATION, Otawara-shi (JP)

(21) Appl. No.: 18/364,000

(22) Filed: Aug. 2, 2023

(30) Foreign Application Priority Data
Aug. 9, 2022 (JP) 2022-127342

(51) Int. Cl. A61B 8/00

(52) U.S. Cl. CPC A61B 8/4477

(57) ABSTRACT

An ultrasonic probe according to an embodiment includes a first transducer group and a second transducer group. The first transducer group includes a plurality of transducers arranged along a first direction. The second transducer group is a second transducer group arranged in an inverted direction with respect to the first transducer group, and includes a plurality of transducers arranged along a second direction different from the first direction.

### CROSS REFERENCE TO RELATED APPLICATIONS

This application is based upon and claims the benefit of priority from the prior Japanese Patent Application No. 2022-127342, filed on Aug. 9, 2022, the entire contents of which are incorporated herein by reference.

### FIELD

Embodiments disclosed in the present specification and drawings relate to an ultrasonic probe and an ultrasonic diagnostic apparatus.

### BACKGROUND

Conventionally, there has been known an intraoperative ultrasonic probe that performs ultrasonic examination by bringing the ultrasonic probe into direct contact with a surface of an organ, a tissue, or the like during laparotomy. There are two types of intraoperative ultrasonic probes: a type in which a drawing-out direction of a cable from a housing is parallel to a scanning direction of ultrasonic wave, and a type in which the drawing-out direction of the cable is orthogonal to the scanning direction.

In the ultrasonic examination during laparotomy, an operation region of the ultrasonic probe is limited. Therefore, it may be difficult to observe different cross sections by rotating the ultrasonic probe about an axis orthogonal to the drawing-out direction of the cable because the cable becomes an obstacle. Furthermore, it is possible to observe different cross sections without rotating the ultrasonic probe by replacing and using the two types of ultrasonic probes described above. However, in this case, complicated work of replacing the ultrasonic probe is required.

### BRIEF DESCRIPTION OF THE DRAWINGS

FIG. 1 is a block diagram illustrating an example of a configuration of an ultrasonic diagnostic apparatus according to a first embodiment.

FIG. 2 is a plan view illustrating an example of a configuration of an ultrasonic probe according to the first embodiment.

FIG. 3 is a bottom view illustrating an example of a configuration of the ultrasonic probe according to the first embodiment.

FIG. 4 is a side view illustrating an example of a configuration of the ultrasonic probe according to the first embodiment.

FIG. 5 is a side view illustrating a configuration of an ultrasonic probe according to a first modification of the first embodiment.

FIG. 6 is a plan view illustrating a configuration of an ultrasonic probe according to a second modification of the first embodiment.

FIG. 7 is a bottom view illustrating a configuration of the ultrasonic probe according to the second modification of the first embodiment.

FIG. 8 is a flowchart illustrating an operation example of the ultrasonic diagnostic apparatus according to the first embodiment.

FIG. 9 is an explanatory diagram for explaining a step of driving a first transducer group in an operation example of the ultrasonic diagnostic apparatus according to the first embodiment.

FIG. 10 is an explanatory diagram for explaining a step of designating a position of a second cross section in an operation example of the ultrasonic diagnostic apparatus according to the first embodiment.

FIG. 11 is an explanatory diagram for explaining a step of calculating a target position of a second transducer group in an operation example of the ultrasonic diagnostic apparatus according to the first embodiment.

FIG. 12 is an explanatory diagram for explaining a step of recording a position of the first transducer group and a target position of the second transducer group in an operation example of the ultrasonic diagnostic apparatus according to the first embodiment.

FIG. 13 is an explanatory diagram for explaining an inversion step of the ultrasonic probe in the operation example of the ultrasonic diagnostic apparatus according to the first embodiment.

FIG. 14 is an explanatory diagram for explaining an example of a step of guiding the movement of the second transducer group in an operation example of the ultrasonic diagnostic apparatus according to the first embodiment.

FIG. 15 is an explanatory diagram for explaining another example of the step of guiding the movement of the second transducer group in the operation example of the ultrasonic diagnostic apparatus according to the first embodiment.

FIG. 16 is an explanatory diagram for explaining a step of driving the second transducer group in an operation example of the ultrasonic diagnostic apparatus according to the first embodiment.

FIG. 17 is an explanatory diagram for explaining a step of displaying an ultrasonic image of a second cross section in an operation example of the ultrasonic diagnostic apparatus according to the first embodiment.

FIG. 18 is a block diagram illustrating an example of a configuration of an ultrasonic diagnostic apparatus according to a second embodiment.

FIG. 19 is a flowchart illustrating an operation example of the ultrasonic diagnostic apparatus according to the second embodiment.

### DETAILED DESCRIPTION

Hereinafter, embodiments of an ultrasonic diagnostic apparatus will be described with reference to the drawings. Note that, in the following description, constituent elements having substantially the same functions and configurations are denoted by the same reference signs, and redundant description will be made only when necessary.

### First Embodiment

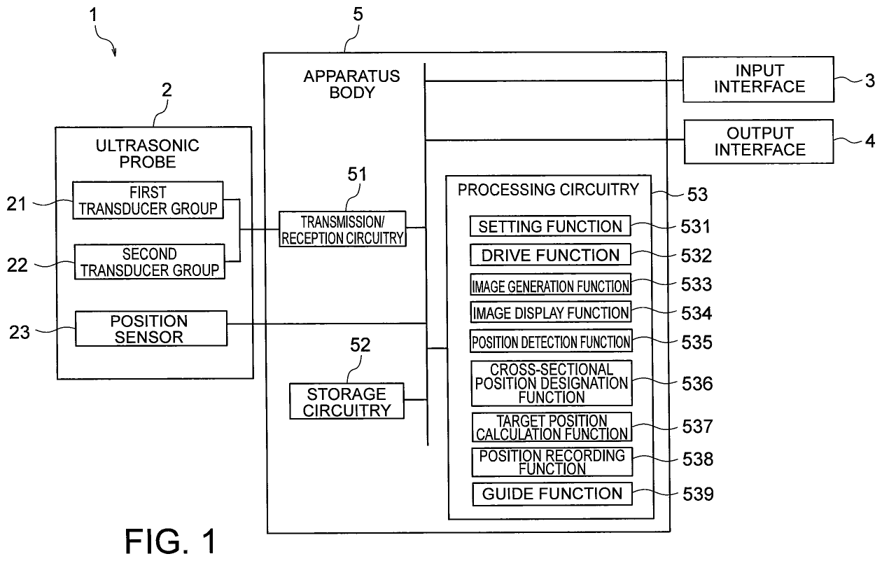

FIG. 1 is a block diagram illustrating an example of a configuration of an ultrasonic diagnostic apparatus 1 according to a first embodiment. As illustrated in FIG. 1, the ultrasonic diagnostic apparatus 1 according to the first embodiment includes an ultrasonic probe 2, an input interface 3, an output interface 4, and an apparatus body 5.

The ultrasonic probe 2 is a device that transmits an ultrasonic wave to a subject and receives a reflected wave signal (echo) of the ultrasonic wave from the subject in order to acquire an ultrasonic image of the subject. The ultrasonic probe 2 can be used, for example, for ultrasonic examination during laparotomy. The ultrasonic probe 2 may be used for applications other than the ultrasonic examination during laparotomy.

The ultrasonic probe 2 includes a first transducer group 21, a second transducer group 22, a position sensor 23, a housing 20, and a cable 6. The position sensor 23 is an example of a position detector.

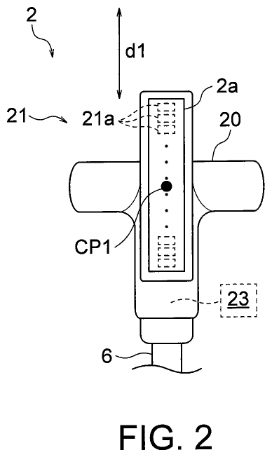

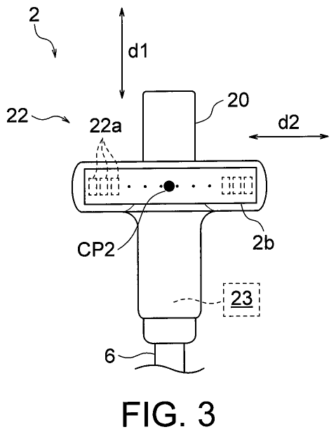

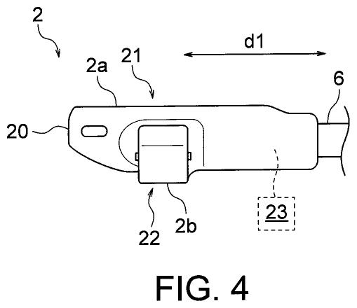

FIG. 2 is a plan view illustrating an example of a configuration of the ultrasonic probe 2 according to the first embodiment. FIG. 3 is a bottom view illustrating an example of a configuration of the ultrasonic probe 2 according to the first embodiment. FIG. 4 is a side view illustrating an example of a configuration of the ultrasonic probe 2 according to the first embodiment. As illustrated in FIG. 2, the first transducer group 21 includes a plurality of transducers 21a arranged along a first direction d1. Note that a size of each of the transducers 21a illustrated in FIG. 2 may be different from the actual size. The transducer 21a is an element that generates an ultrasonic wave by vibrating by application of a voltage. Furthermore, the transducer 21a is an element that receives a reflected wave signal from the subject and converts the reflected wave signal into an electric signal. The transducer 21a is disposed inside the housing 20. In the example illustrated in FIGS. 2 and 3, the housing 20 has a substantially cross shape in plan view. The transducer 21a is provided with an electrode for applying a voltage. The transducer 21a may be made of, for example, lead zirconate titanate (PZT) or polyvinylidene fluoride (PVDF). An acoustic matching layer and an acoustic lens may be disposed on a surface of the first transducer group 21. A backing material may be disposed on a back surface of the first transducer group 21. The acoustic matching layer is also called a A/4 layer, and is a layer for efficiently transmitting and receiving ultrasonic waves by reducing an impedance difference between the transducer 21a and a living body. The acoustic lens is a structure for reducing friction with a living body surface at the time of examination and for improving a slice resolution by converging an ultrasonic beam. The backing material has a structure that absorbs rearward ultrasonic waves and shortens a pulse width of forward ultrasonic waves.

In the example illustrated in FIG. 2, the transducers 21a of the first transducer group 21 are linearly arranged along the first direction d1. Specifically, the transducers 21a of the first transducer group 21 are arranged along the cable 6 electrically connected to the first transducer group 21 and the second transducer group 22. More specifically, the transducers 21a of the first transducer group 21 are arranged along a drawing-out direction of the cable 6 from the housing 20 (that is, a head part). The first transducer group 21 is driven by a linear electron scanning method in which the first direction d1 is set as a scanning direction. The linear electron scanning method is a method in which two or more adjacent transducers 21a in the first transducer group 21 are selectively driven, and the two or more transducers 21a to be driven are scanned while being sequentially shifted in the scanning direction.

As illustrated in FIGS. 3 and 4, the second transducer group 22 is disposed in an inverted direction with respect to the first transducer group 21. That is, in the ultrasonic probe 2, a second acoustic surface 2b which is an emitting surface of an ultrasonic wave by the second transducer group 22 is provided in a direction opposite to a first acoustic surface 2a which is an emitting surface of an ultrasonic wave by the first transducer group 21. The first acoustic surface 2a may be, for example, a surface of an acoustic lens provided on the first transducer group 21. The second acoustic surface 2b may be, for example, a surface of an acoustic lens provided on the second transducer group 22. The second transducer group 22 includes a plurality of transducers 22a arranged in a second direction d2. Note that a size of each of the transducers 22a illustrated in FIG. 3 may be different from the actual size. The second direction d2 is a direction different from the first direction d1. For example, the second direction d2 is a direction intersecting the first direction d1. More specifically, the second direction d2 is a direction intersecting the first direction d1 when viewed from a thickness direction of the ultrasonic probe 2, that is, a normal direction of the first acoustic surface 2a (in other words, a transducer surface). In the example illustrated in FIG. 3, the second direction d2 is a direction orthogonal to the first direction d1. A specific configuration of the transducers 22a of the second transducer group 22 is similar to that of the first transducer group 21. Furthermore, similarly to the first transducer group 21, the second transducer group 22 is driven by the linear electron scanning method in which the second direction d2 is set as the scanning direction.

Since the first transducer group 21 and the second transducer group 22 are arranged in the inverted direction, the ultrasonic probe 2 drives one transducer group to scan a cross section of the subject, and then, is used in an inverted state, so that the other transducer group can be driven to scan different cross sections of the subject.

The position sensor 23 is a sensor for detecting positions of the first transducer group 21 and the second transducer group 22. The position sensor 23 is electrically connected to the apparatus body 5 via the cable 6, for example. In the example illustrated in FIGS. 2 to 4, the position sensor 23 is disposed inside the housing 20. The position sensor 23 is not limited to being disposed inside the housing 20, and may be fixed to the outside of the housing 20.

The position sensor 23 may be used to detect one representative position of the first transducer group 21 as the position of the first transducer group 21. For example, the position sensor 23 may be used to detect a center position CP1 (see FIG. 2) of the first transducer group 21 in the first direction d1 as the position of the first transducer group 21. The center position CP1 of the first transducer group 21 in the first direction d1 may be detected as a relative position with respect to the position of the position sensor 23 on the basis of a known correspondence between the CP1 and the position of the position sensor 23. Hereinafter, the center position CP1 of the first transducer group 21 in the first direction d1 may be referred to as a position CP1 of the first transducer group 21.

Furthermore, the position sensor 23 may be used to detect one representative position of the second transducer group 22 as the position of the second transducer group 22. For example, the position sensor 23 may be used to detect a center position CP2 (see FIG. 3) of the second transducer group 22 in the second direction d2 as the position of the second transducer group 22. The center position CP2 of the second transducer group 22 in the second direction d2 may be detected as a relative position with respect to the position of the position sensor 23 on the basis of a known correspondence between the CP2 and the position of the position sensor 23. Hereinafter, the center position CP2 of the second transducer group 22 in the second direction d2 may be referred to as a position CP2 or a current position CP2 of the second transducer group 22.

The position CP2 of the second transducer group 22 may have a positional relationship of facing the position CP1 of the first transducer group 21 in the thickness direction of the ultrasonic probe 2. That is, the position CP2 of the second transducer group 22 may have a positional relationship matching the position CP1 of the first transducer group 21 before inversion when the ultrasonic probe 2 is inverted without being horizontally moved. The position CP2 of the second transducer group 22 is not limited to the facing positional relationship in the thickness direction of the ultrasonic probe 2, and may have an arbitrary positional relationship with respect to the position CP1 of the first transducer group 21.

For example, the position sensor 23 may be configured to receive a signal transmitted from a transmitter provided in the apparatus body 5 and output an electric signal corresponding to the position of the position sensor 23 to the apparatus body 5. In this case, the apparatus body 5 (specifically, a position detection function 535 of a processing circuitry 53 to be described later) calculates the positions of the first transducer group 21 and the second transducer group 22 with a predetermined position as an origin based on the electric signal from the position sensor 23. For example, the position sensor 23 may be a receiver that receives a magnetic signal transmitted from a magnetic generator as a transmitter and outputs an electric signal according to a relative position with respect to the magnetic generator. That is, the position sensor 23 (receiver) may constitute a magnetic sensor together with a transmitter. The configuration is not limited to the configuration requiring a transmitter of the apparatus body 5, and the position sensor 23 may be configured to output an electric signal corresponding to the position of the position sensor 23 to the apparatus body 5 (that is, the position detection function 535) without requiring a transmitter. For example, the position sensor 23 may be a gyro sensor, an acceleration sensor, a global positioning system (GPS) sensor, or the like.

The input interface 3 receives various instructions and information input operations from an operator. Specifically, the input interface 3 converts an input operation received from the operator into an electric signal and outputs the electric signal to the apparatus body 5. For example, the input interface 3 is realized by a trackball, a switch button, a mouse, a keyboard, a touch pad that performs an input operation by touching an operation surface, a touch screen in which a display screen and the touch pad are integrated, a non-contact input circuitry using an optical sensor, a sound input circuitry, and the like. Note that the input interface 3 is not limited to one including physical operation components such as a mouse and a keyboard. For example, an electric signal processing circuitry that receives an electric signal corresponding to an input operation from an external input device provided separately from the apparatus and outputs the electric signal to a control circuitry is also included in the example of the input interface 3.

The output interface 4 outputs various types of information. For example, the output interface 4 includes a display. The display converts information and image data transmitted from the apparatus body 5 into an electric signal for display and outputs the electric signal. The display is realized by a liquid crystal monitor, a cathode ray tube (CRT) monitor, a touch panel, and the like. The output interface 4 may include a speaker. The speaker outputs a predetermined sound such as a beep sound to notify the operator of the processing status of the apparatus body 5.

The apparatus body 5 includes a transmission/reception circuitry 51, a storage circuitry 52, and a processing circuitry 53. The storage circuitry 52 is an example of a memory.

The transmission/reception circuitry 51 is a circuitry that switches and drives the first transducer group 21 and the second transducer group 22 under the control of the processing circuitry 53. That is, the transmission/reception circuitry 51 is a circuitry that selects and drives one of the first transducer group 21 and the second transducer group 22. The transmission/reception circuitry 51 is electrically connected to the first transducer group 21 and the second transducer group 22 via the cable 6.

The transmission/reception circuitry 51 includes, for example, a pulse generator, a transmission delay unit, a pulser, and the like in order to supply a drive signal to the ultrasonic probe 2. The pulse generator repeatedly generates rate pulses for forming transmission ultrasonic waves at a predetermined rate frequency. Furthermore, the transmission delay unit focuses the ultrasonic wave generated from the ultrasonic probe 2 in a beam shape and gives a delay time for each transducer necessary for determining transmission directivity to each rate pulse generated by the pulse generator. The pulser applies a drive signal (drive pulse) to the ultrasonic probe 2 at timing based on the rate pulse. That is, the transmission delay unit arbitrarily adjusts a transmission direction of the ultrasonic wave transmitted from the transducer surface (that is, the acoustic surface) by changing the delay time given to each rate pulse.

Furthermore, the transmission/reception circuitry 51 includes, for example, a preamplifier, an analog/digital (A/D) converter, a reception delay unit, an adder, and the like in order to perform various processing on the reflected wave signal received by the ultrasonic probe 2 to generate reflected wave data. The preamplifier amplifies the reflected wave signal for each channel. The A/D converter A/D converts the amplified reflected wave signal. The reception delay unit gives a delay time necessary for determining reception directivity. The adder performs addition processing of the reflected wave signal processed by the reception delay unit to generate reflected wave data. By the addition processing of the adder, a reflection component from a direction according to the reception directivity of the reflected wave signal is emphasized, and a comprehensive beam of ultrasonic transmission and reception is formed by the reception directivity and the transmission directivity. The form of the output signal from the transmission/reception circuitry 51 can be selected from various forms such as a case where the output signal is a signal including phase information called a radio frequency (RF) signal and a case where the output signal is amplitude information after envelope detection processing.

In the example illustrated in FIG. 1, the transmission/reception circuitry 51 is disposed in the apparatus body 5. The transmission/reception circuitry 51 is not limited to being disposed in the apparatus body 5, and at least a part of the transmission/reception circuitry 51 may be disposed inside the housing 20 of the ultrasonic probe 2.

The storage circuitry 52 is a non-transitory storage device that stores various types of information, and is, for example, a hard disk drive (HDD), an optical disk, a solid state drive (SSD), an integrated circuitry storage device, or the like. The storage circuitry 52 stores, for example, a control program for controlling the ultrasonic diagnostic apparatus 1 and various data used for executing the control program. The storage circuitry 52 may be a drive device that reads and writes various types of information from and to a portable storage medium such as a compact disc (CD), a digital versatile disc (DVD), and a flash memory, a semiconductor memory element such as a random access memory (RAM), or the like, in addition to the HDD, the SSD, and the like.

The processing circuitry 53 is a circuitry that controls the entire operation of the ultrasonic diagnostic apparatus 1 according to an electric signal of an input operation input from the input interface 3. For example, the processing circuitry 53 includes a setting function 531, a drive function 532, an image generation function 533, an image display function 534, a position detection function 535, a cross-sectional position designation function 536, a target position calculation function 537, a position recording function 538, and a guide function 539. The setting function 531 is an example of a setting unit. The drive function 532 is an example of a drive unit. The image generation function 533 is an example of an image generation unit. The image display function 534 is an example of an image display unit. The position detection function 535 is an example of a position detector. The cross-sectional position designation function 536 is an example of a cross-sectional position designation unit. The target position calculation function 537 is an example of a target position calculation unit. The position recording function 538 is an example of a position recording unit. The guide function 539 is an example of a guide part.

Here, for example, each processing function executed by the setting function 531, the drive function 532, the image generation function 533, the image display function 534, the position detection function 535, the cross-sectional position designation function 536, the target position calculation function 537, the position recording function 538, and the guide function 539, which are components of the processing circuitry 53 illustrated in FIG. 1, is recorded in the storage circuitry 52 in the form of a program executable by a computer. The processing circuitry 53 is, for example, a processor. The processor constituting the processing circuitry 53 reads each program from the storage circuitry 52 and executes the program to implement a function corresponding to each read program. In other words, the processing circuitry 53 in a state of reading each program has each function illustrated in the processing circuitry 53 of FIG. 1.

Note that FIG. 1 illustrates a case where each processing function of the setting function 531, the drive function 532, the image generation function 533, the image display function 534, the position detection function 535, the cross-sectional position designation function 536, the target position calculation function 537, the position recording function 538, and the guide function 539 is realized by a single processing circuitry 53, but the embodiment is not limited thereto. For example, the processing circuitry 53 may be configured by combining a plurality of independent processors, and each processor may implement each processing function by executing each program. Furthermore, each processing function of the processing circuitry 53 may be implemented by being appropriately distributed or integrated into a single or a plurality of processing circuitries.

The setting function 531 sets one of the first transducer group 21 and the second transducer group 22 as a transducer group to be driven. In the first embodiment, the setting function 531 sets one of the first transducer group 21 and the second transducer group 22 as a transducer group to be driven according to an input operation of the operator.

The drive function 532 drives one transducer group set as the transducer group driven by the setting function 531 out of the first transducer group 21 and the second transducer group 22.

The image generation function 533 generates an ultrasonic image of the subject on the basis of a reflected wave signal of an ultrasonic wave received by the transducer group driven by the drive function 532. Specifically, when the first transducer group 21 is driven by the drive function 532, the image generation function 533 generates an ultrasonic image of a first cross section of the subject corresponding to the first transducer group 21. Furthermore, when the second transducer group 22 is driven by the drive function 532, the image generation function 533 generates an ultrasonic image of a second cross section of the subject corresponding to the second transducer group 22. The second cross section is a cross section different from the first cross section. More specifically, the second cross section is a cross section intersecting the first cross section. The second cross section may be a cross section orthogonal to the first cross section.

The image display function 534 displays the ultrasonic image of the subject generated by the image generation function 533 via the output interface 4. Specifically, the image display function 534 displays the ultrasonic image of the first cross section of the subject when the ultrasonic image of the first cross section of the subject is generated by the image generation function 533. Furthermore, the image display function 534 displays the ultrasonic image of the second cross section of the subject when the ultrasonic image of the second cross section of the subject is generated by the image generation function 533.

The position detection function 535 detects positions of the first transducer group 21 and the second transducer group 22 via the position sensor 23. Specifically, the position detection function 535 acquires an electric signal corresponding to the position of the position sensor 23 from the position sensor 23, and calculates (that is, detects) the positions of the first transducer group 21 and the second transducer group 22 on the basis of the acquired electric signal. More specifically, the position detection function 535 detects the position CP1 of the first transducer group 21 and the position CP2 of the second transducer group 22 as three-dimensional coordinates (XYZ coordinates) with a predetermined position as an origin. For example, the position detection function 535 continuously detects the positions of the first transducer group 21 and the second transducer group 22 during the ultrasonic examination.

In a case where the ultrasonic image of the first cross section of the subject is generated by driving the first transducer group 21 and the generated ultrasonic image of the first cross section is displayed, the cross-sectional position designation function 536 receives a designation operation of the position of the second cross section of the subject on the displayed ultrasonic image of the first cross section. The cross-sectional position designation function 536 receives the designation operation of the position of the second cross section via the input interface 3.

The target position calculation function 537 calculates a target position of the second transducer group 22 corresponding to the position of the second cross section designated by the designation operation of the position of the second cross section.

When the ultrasonic image of the first cross section is generated by the drive of the first transducer group 21, and the position of the first transducer group 21 when the ultrasonic image of the first cross section is generated is detected by the position detection function 535, the position recording function 538 records the detected position of the first transducer group 21 in the storage circuitry 52.

For example, the position recording function 538 may record the position CP1 of the first transducer group 21 when the ultrasonic image of the first cross section is generated as a new origin of the XYZ coordinates in the storage circuitry 52. That is, the position recording function 538 may convert the XYZ coordinates such that the position CP1 of the first transducer group 21 when the ultrasonic image of the first cross section is generated becomes the new origin of the XYZ coordinates. Accordingly, the position recording function 538 may record the target position of the second transducer group 22 in the storage circuitry 52 as a target position based on the new origin CP1 of the XYZ coordinates. As a result, even after the inversion of the ultrasonic probe 2, the current position CP2 and the target position of the second transducer group 22 can be associated with the new origin located on the first cross section. That is, after the inversion of the ultrasonic probe 2, the position detection function 535 can detect the current position CP2 of the second transducer group 22 as a displacement amount from the new origin located on the first cross section. Therefore, it is possible to guide the movement of the second transducer group 22 to the target position on the first cross section by the guide function 539 described later in an easy-to-understand manner for the operator.

When the ultrasonic image of the second cross section of the subject corresponding to the second transducer group 22 is generated by driving the second transducer group 22, the guide function 539 uses the position of the first transducer group 21 recorded in the storage circuitry 52 as a reference of the position of the second transducer group 22 to guide the movement of the second transducer group 22 to the position where the ultrasonic image of the second cross section is acquired. Specifically, the guide function 539 guides the movement of the second transducer group 22 according to the position of the second cross section designated by the designation operation of the second cross section. More specifically, the guide function 539 guides the movement of the second transducer group 22 to the target position calculated by the target position calculation function 537, thereby guiding the movement of the second transducer group 22 according to the position of the second cross section. More specifically, the guide function 539 guides the movement of the second transducer group 22 by at least one of image display and sound output via the output interface 4. The guide function 539 may use the new origin of the XYZ coordinates described above as a reference of the position of the second transducer group 22 to guide the movement of the second transducer group 22 to the target position with the origin as a reference.

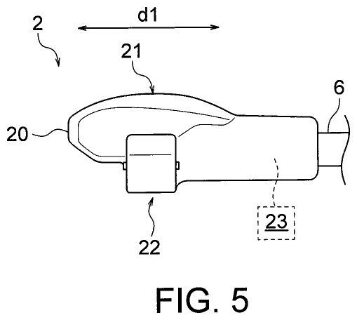

FIG. 5 is a side view illustrating a configuration of an ultrasonic probe 2 according to a first modification of the first embodiment. In the examples illustrated in FIGS. 2 to 4, a scanning method of the first transducer group 21 is the same linear electronic scanning method as the scanning method of the second transducer group 22. The scanning method is not limited to the same as the scanning method of the second transducer group 22, and the scanning method of the first transducer group 21 may be different from the scanning method of the second transducer group 22. Specifically, as illustrated in FIG. 5, the scanning method of the first transducer group 21 may be a convex electronic scanning method, and the scanning method of the second transducer group 22 may be a linear electronic scanning method. In the example illustrated in FIG. 5, the transducers 21a of the first transducer group 21 are arranged in a convex shape (that is, a circular arc shape) along the first direction d1. Similarly to the linear electronic scan method, the convex electronic scan method is also a method in which two or more adjacent transducers 21a are selectively driven in the first transducer group 21, and the two or more transducers 21a to be driven are scanned while being sequentially shifted in the first direction d1. According to the convex electronic scan method, it is possible to observe a wide range in a deep portion while suppressing a contact area with the subject. Alternatively, the scanning method of the first transducer group 21 may be a sector electronic scanning method. The sector electronic scan method is a scan method capable of freely changing a direction of the transmitted ultrasonic wave by shifting the drive timing of the linearly arranged transducers. According to the sector electronic scan method, a wide range of observation is possible while suppressing the contact area with the subject. The scanning method of the first transducer group 21 and the second transducer group 22 may be any of the linear electronic scanning method, the convex electronic scanning method, and the sector electronic scanning method. Note that the arrangement direction d1 of the first transducer group 21 of the convex electronic scanning method and the arrangement direction d2 of the second transducer group 22 of the linear electronic scanning method may not intersect as long as they are different directions. The same applies to a combination of the transducer groups 21 and 22 of other scanning methods.

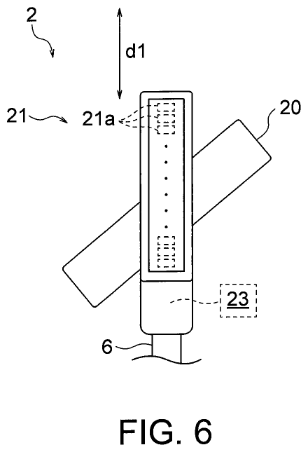

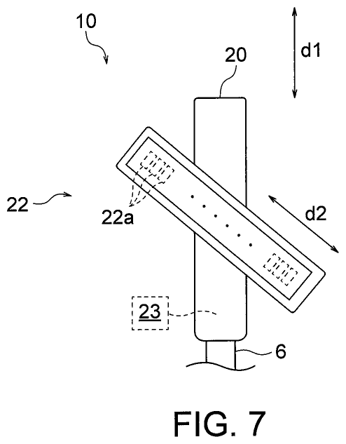

FIG. 6 is a plan view illustrating a configuration of an ultrasonic probe 2 according to a second modification of the first embodiment. FIG. 7 is a bottom view illustrating a configuration of the ultrasonic probe 2 according to the second modification of the first embodiment. In the example illustrated in FIGS. 2 to 4, the second direction d2 is a direction orthogonal to the first direction d1. On the other hand, as illustrated in FIGS. 6 and 7, the second direction d2 may be a direction intersecting the first direction d1 at an angle other than 90°.

Next, an operation example of the ultrasonic diagnostic apparatus 1 according to the first embodiment configured as described above will be described. Note that, in the following operation example, an example will be given in which both the scanning methods of the first transducer group 21 and the second transducer group 22 are the linear electronic scanning method, but the following operation example can also be applied to a combination of other scanning methods described above.

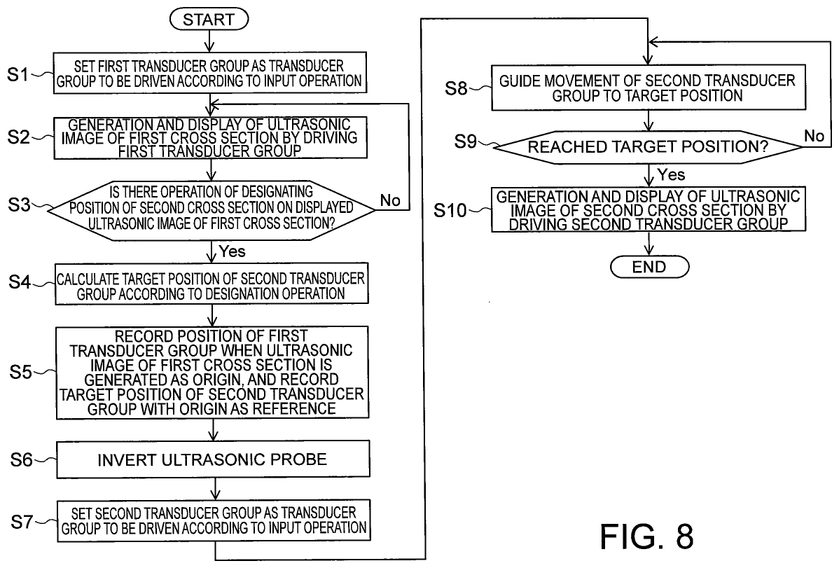

FIG. 8 is a flowchart illustrating an operation example of the ultrasonic diagnostic apparatus 1 according to the first embodiment. First, as illustrated in FIG. 8, the setting function 531 sets the first transducer group 21 as a transducer group to be driven (step S1). Specifically, the setting function 531 sets the first transducer group 21 as a transducer group to be driven in response to an input operation for setting the first transducer group 21 as a transducer group to be driven by the operator via the input interface 3. A specific mode for setting the transducer group to be driven by the setting function 531 is not particularly limited, and may be realized, for example, by providing a switch for switching the transducer group to which a drive signal from a power supply circuitry is applied between the first transducer group 21 and the second transducer group 22 in the transmission/reception circuitry 51. In this case, the setting function 531 may cause the switch to connect the first transducer group 21 to the power supply circuitry and disconnect the second transducer group 22 from the power supply circuitry, thereby setting the first transducer group 21 as the transducer group to be driven. Alternatively, a switch or a function equivalent thereto may be provided in the processing circuitry 53.

After the first transducer group 21 is set as the transducer group to be driven, the drive function 532 drives the first transducer group 21 according to the electronic scanning method of the first transducer group 21 via the transmission/reception circuitry 51. By being driven by the drive function 532, the first transducer group 21 scans the first cross section of the subject along the first direction d1 with ultrasonic waves. For example, after the first transducer group 21 is moved to a predetermined position by the operator, the drive function 532 may start driving the first transducer group 21 in response to an input operation for instructing the drive of the first transducer group 21 performed by the operator. Alternatively, the drive function 532 may start driving the first transducer group 21 in response to setting the first transducer group 21 as a transducer group to be driven. The image generation function 533 generates an ultrasonic image of the first cross section based on a scan result by the first transducer group 21. The image display function 534 displays the ultrasonic image of the first cross section via the output interface 4 (step S2).

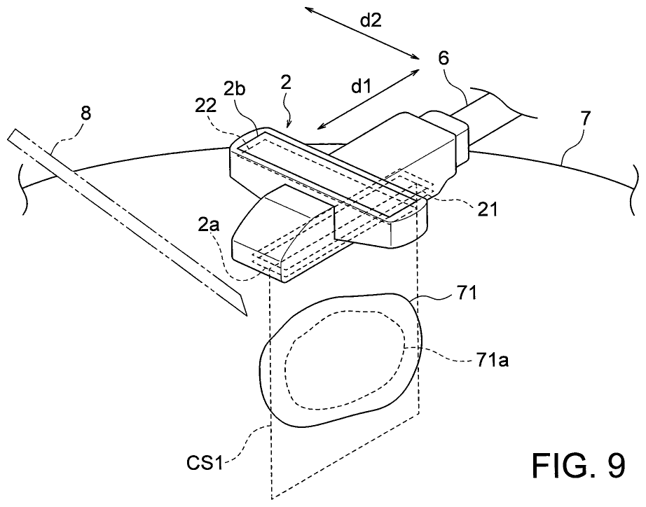

FIG. 9 is an explanatory diagram for explaining a driving process of the first transducer group 21 in an operation example of the ultrasonic diagnostic apparatus 1 according to the first embodiment. As illustrated in FIG. 9, the ultrasonic probe 2 is used by bringing the first acoustic surface 2a corresponding to the first transducer group 21 into direct contact with an internal surface 7 of a subject in an open abdomen state such as an organ and a tissue. Note that, in FIG. 9, the first transducer group 21 and the second transducer group 22 are illustrated in a simplified manner. The ultrasonic probe 2 is used, for example, for examination or treatment using a puncture needle 8. The ultrasonic probe 2 may be used for examination without using the puncture needle 8.

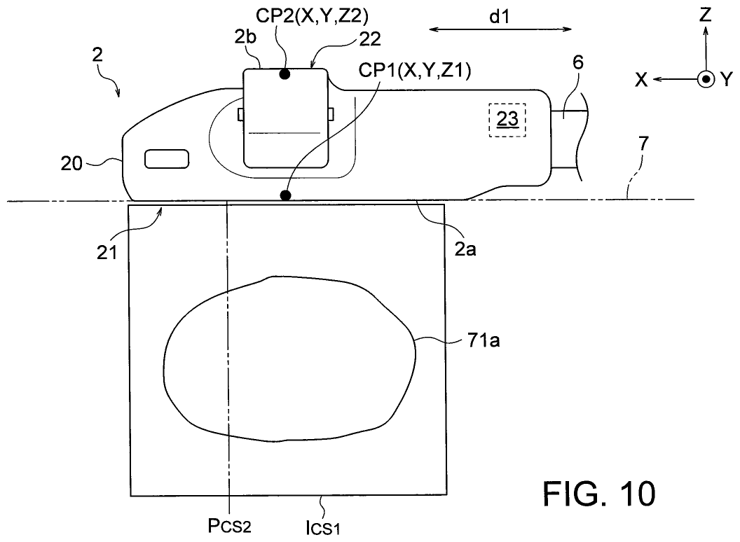

In the example illustrated in FIG. 9, the first transducer group 21 scans a first cross section CS1 including an observation target 71 such as a lesion with ultrasonic waves. The image generation function 533 generates an ultrasonic image I_{CS1} (see FIG. 10) of the first cross section CS1 including a cross section 71a of the observation target 71 on the basis of a scan result of the first transducer group 21.

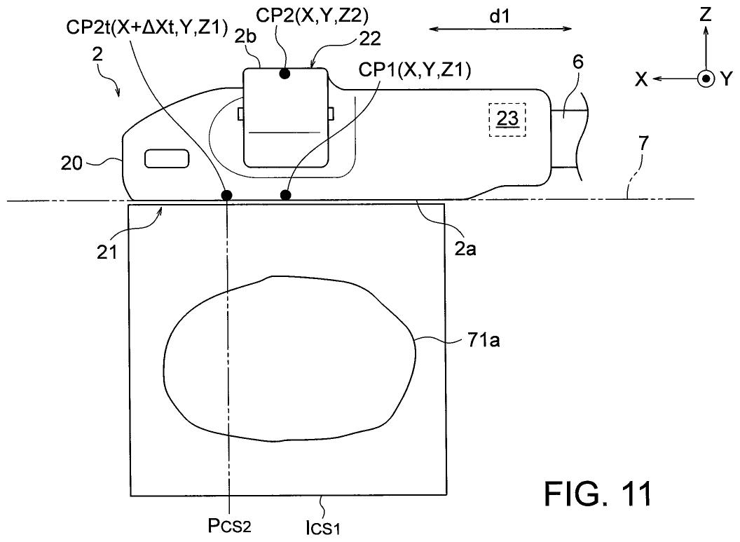

FIG. 10 is an explanatory diagram for explaining a step of designating the position of the second cross section in the operation example of the ultrasonic diagnostic apparatus 1 according to the first embodiment. As illustrated in FIG. 10, the image display function 534 displays, via the output interface 4, the ultrasonic image I_{CS1} of the first cross section CS1 generated by the image generation function 533. Note that FIG. 10 illustrates a side view of the ultrasonic probe 2 together with the ultrasonic image I_{CS1} in order to illustrate a correspondence relationship between a position on the ultrasonic image I_{CS1} and a position on the ultrasonic probe 2 (The same applies to FIGS. 11 to 13).

For easy understanding, in the example illustrated in FIG. 10, an X axis is defined along the first direction d1. Furthermore, a Y axis is defined along the second direction d2. Furthermore, a Z axis is defined along the thickness direction of the ultrasonic probe 2. Furthermore, the position CP1 of the first transducer group 21 and the position CP2 of the second transducer group 22 have a positional relationship of facing (That is, the X coordinate and the Y coordinate coincide with each other.) each other in the thickness direction of the ultrasonic probe 2. Furthermore, the internal surface 7 of the subject is considered to be parallel to an XY plane. The actual XYZ coordinates may be different from the example illustrated in FIG. 10.

In the example illustrated in FIG. 10, the position of the first transducer group 21 when the ultrasonic image I_{CS1} of the first cross section CS1 is generated is CP1 (X, Y, Z1). Furthermore, the position of the second transducer group 22 when the ultrasonic image I_{CS1} of the first cross section CS1 is generated is CP2 (X, Y, Z2). Note that, at the time point of FIG. 10, the position CP1 (X, Y, Z1) of the first transducer group 21 is not the origin of the XYZ coordinates, and a predetermined position set by default in the apparatus body 5 is the origin of the XYZ coordinates.

After the ultrasonic image of the first cross section is displayed, as illustrated in FIG. 8, the cross-sectional position designation function 536 determines whether or not the position of the second cross section is designated by the operator on the displayed ultrasonic image of the first cross section (step S3). Specifically, the cross-sectional position designation function 536 determines whether or not an operation of designating the position of the second cross section is input from the input interface 3. The operation of designating the position of the second cross section may be, for example, an operation of inputting the X coordinate of the second cross section. The operation of designating the position of the second cross section is not limited to the operation of inputting the X coordinate of the second cross section, and the operation of designating the position of the second cross section may be an operation of instructing the position of the second cross section on the ultrasonic image of the first cross section with a pointer or the like. FIG. 10 illustrates a position P_{CS2} of the second cross section designated on the ultrasonic image I_{CS1} of the first cross section.

In a case where the position of the second cross section is designated (step S3: Yes), as illustrated in FIG. 8, the target position calculation function 537 calculates a target position of the second transducer group 22 (step S4). For example, the target position calculation function 537 reads the correspondence relationship between the coordinates (image coordinates) of the ultrasonic image and the coordinates (spatial coordinates) of the second transducer group 22 stored in the storage circuitry 52, and calculates the target position of the second transducer group 22 corresponding to the designated position of the second cross section based on the read correspondence relationship.

FIG. 11 is an explanatory diagram for explaining a step of calculating the target position of the second transducer group 22 in the operation example of the ultrasonic diagnostic apparatus 1 according to the first embodiment. In the example illustrated in FIG. 11, the target position calculation function 537 calculates a target position CP2t (X+ΔXt, Y, Z1) of the second transducer group 22 corresponding to the designated position P_{CS2} of the second cross section.

On the other hand, in a case where the position of the second cross section is not designated (step S3: No), as illustrated in FIG. 8, generation and display of the ultrasonic image of the first cross section by driving the first transducer group 21 are repeated (step S2).

After the target position of the second transducer group 22 is calculated, the position recording function 538 records the position of the first transducer group 21 when the ultrasonic image of the first cross section is generated as the origin in the storage circuitry 52. Furthermore, the position recording function 538 records the target position of the second transducer group 22 with the origin as a reference in the storage circuitry 52 (step S5).

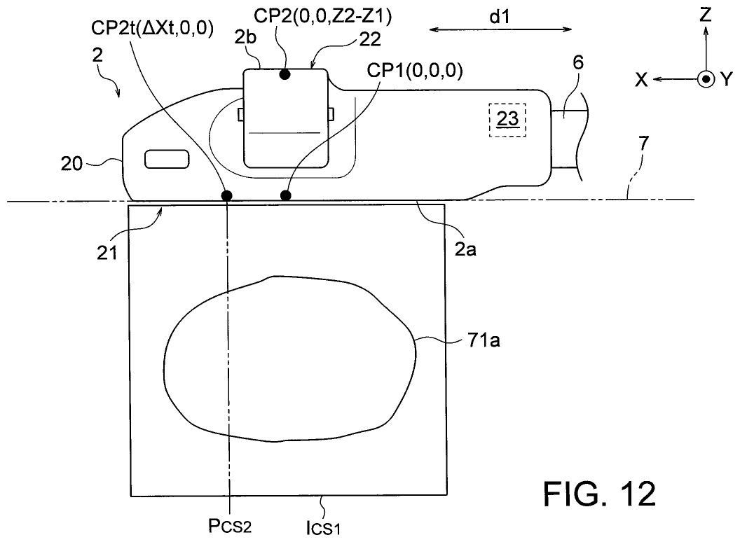

FIG. 12 is an explanatory diagram for explaining a step of recording the position of the first transducer group 21 and the target position of the second transducer group 22 in the operation example of the ultrasonic diagnostic apparatus according to the first embodiment. In the example illustrated in FIG. 12, the position recording function 538 records the position CP1 (X, Y, Z1) of the first transducer group 21 when the ultrasonic image of the first cross section is generated in the storage circuitry 52 as a new origin CP1 (0, 0, 0) of the XYZ coordinates. That is, the position recording function 538 converts the XYZ coordinates such that the position CP1 (X, Y, Z1) of the first transducer group 21 when the ultrasonic image of the first cross section is generated becomes the new origin of the XYZ coordinates. Furthermore, in the example illustrated in FIG. 12, the position recording function 538 records the target position CP2t (X+ΔXt, Y, Z1) of the second transducer group 22 in the storage circuitry 52 as a target position CP2t (ΔXt, 0, 0) with the new origin CP1 (0, 0, 0) as a reference. Note that, in the description to be described later of the operation of the ultrasonic probe 2 after the inversion, the new origin of the XYZ coordinates may be referred to as an origin O (0,0,0).

After the position of the first transducer group 21 when the ultrasonic image of the first cross section is generated is recorded as the origin and the target position of the second transducer group 22 with the origin as a reference is recorded, as illustrated in FIG. 8, the operator inverts the ultrasonic probe 2 (step S6). The inversion of the ultrasonic probe 2 may be performed according to a guide (for example, guidance by at least one of an image and a sound) by which the guide function 539 instructs the operator a timing at which the ultrasonic probe 2 is inverted. Alternatively, the inversion of the ultrasonic probe 2 may be performed according to the determination of the operator.

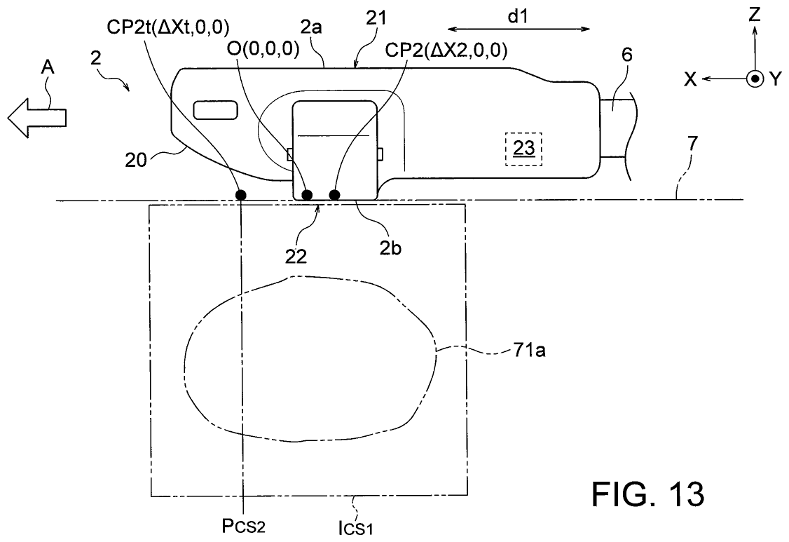

FIG. 13 is an explanatory diagram for explaining an inversion step of the ultrasonic probe 2 in the operation example of the ultrasonic diagnostic apparatus 1 according to the first embodiment. By inverting the ultrasonic probe 2, as illustrated in FIG. 13, the operator brings the second acoustic surface 2b corresponding to the second transducer group 22 into direct contact with the internal surface 7 of the subject.

After the ultrasonic probe 2 is inverted, as illustrated in FIG. 8, the setting function 531 sets the second transducer group 22 as a transducer group to be driven (step S7). Specifically, the setting function 531 sets the second transducer group 22 as a transducer group to be driven in response to an input operation for setting the second transducer group 22 as a transducer group to be driven by the operator via the input interface 3. For example, the setting function 531 may cause the switch of the transmission/reception circuitry 51 to disconnect the first transducer group 21 from the power supply circuitry and connect the second transducer group 22 to the power supply circuitry, thereby setting the second transducer group 22 as the transducer group to be driven.

After the second transducer group 22 is set as the transducer group to be driven, the guide function 539 guides the movement of the second transducer group 22 to the target position (step S8). Specifically, the guide function 539 reads the target position of the second transducer group 22 from the storage circuitry 52. Then, the guide function 539 guides the movement of the second transducer group 22 to the read target position by at least one of image display and sound output.

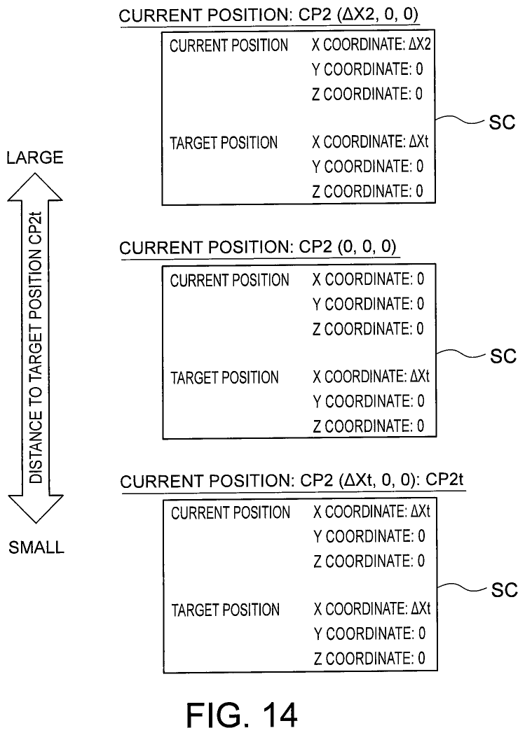

FIG. 14 is an explanatory diagram for explaining an example of a step of guiding the movement of the second transducer group 22 in the operation example of the ultrasonic diagnostic apparatus 1 according to the first embodiment. In the example illustrated in FIG. 14, the guide function 539 guides the movement of the second transducer group 22 by a screen SC displayed via the output interface 4. Specifically, the guide function 539 displays the XYZ coordinates indicating the current position CP2 of the second transducer group 22 and the XYZ coordinates indicating the target position CP2t of the second transducer group 22 on the screen SC in a comparable display mode. For example, the guide function 539 displays the XYZ coordinates indicating the current position CP2 of the second transducer group 22 and the XYZ coordinates indicating the target position CP2t of the second transducer group 22 side by side on the screen SC. The guide function 539 changes the XYZ coordinates of the second transducer group 22 displayed on the screen SC according to a change in the current position CP2 of the second transducer group 22 detected by the position detection function 535.

More specifically, in the example illustrated in FIG. 14, as illustrated in FIG. 13, it is assumed that the position of the second transducer group 22 is CP2 (ΔX2, 0, 0) with the origin O (0, 0, 0) as a reference at the beginning when the ultrasonic probe 2 is inverted. That is, in the example illustrated in FIGS. 13 and 14, CP2 (ΔX2, 0, 0) of the second transducer group 22 at the beginning of the inversion of the ultrasonic probe 2 is on the first cross section. When the ultrasonic probe 2 is moved in the X-axis direction along an arrow A from the state of FIG. 13, as illustrated in FIG. 14, the current position CP2 of the second transducer group 22 reaches the target position CP2t (ΔXt, 0, 0) after passing through the origin O (0, 0, 0). In the example illustrated in FIG. 14, the current position CP2 and the target position (ΔXt, 0, 0) of the second transducer group 22 are displayed as a displacement amount based on the origin O (0, 0, 0) on the first cross section. Therefore, the operator can easily recognize how much the current position CP2 of the second transducer group 22 deviates from the first cross section and the target position CP2t. As a result, the movement of the second transducer group 22 to the target position CP2t on the first cross section can be guided so that the operator can easily understand the movement.

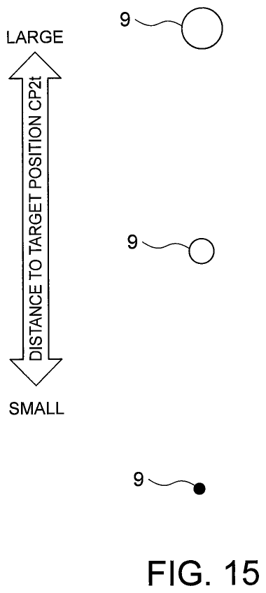

FIG. 15 is an explanatory diagram for explaining another example of the step of guiding the movement of the second transducer group 22 in the operation example of the ultrasonic diagnostic apparatus 1 according to the first embodiment. The guide function 539 is not limited to the mode illustrated in FIG. 14, and may guide the movement of the second transducer group 22 to the target position CP2t by displaying, on the screen SC, a mark 9 whose size decreases as a distance between the current position CP2 and the target position CP2t of the second transducer group 22 decreases as illustrated in FIG. 15. In the example illustrated in FIG. 15, the shape of the mark 9 is circular. The guide function 539 is not limited to the circular mark 9, and may guide the movement of the second transducer group 22 by displaying a mark having a shape other than the circular shape on the screen SC. Furthermore, in a case where the current position CP2 of the second transducer group 22 is located on the first cross section, the color of the mark 9 may be changed.

The guide function 539 is not limited to the modes illustrated in FIGS. 14 and 15, and may guide the movement of the second transducer group 22 using sound by generating a beep sound when the current position CP2 of the second transducer group 22 reaches the target position CP2t, for example.

Next, as illustrated in FIG. 8, the drive function 532 determines whether or not the second transducer group 22 has reached the target position by the guide of the guide function 539 (step S9). That is, the drive function 532 determines whether or not the position CP2 of the second transducer group 22 detected by the position detection function 535 matches the target position CP2t of the second transducer group 22.

In a case where the second transducer group 22 has reached the target position (step S9: Yes), the drive function 532 drives the second transducer group 22 via the transmission/reception circuitry 51 according to the electronic scanning method of the second transducer group 22. By being driven by the drive function 532, the second transducer group 22 scans the second cross section of the subject along the second direction d2 with ultrasonic waves. The image generation function 533 generates an ultrasonic image of the second cross section based on a scan result by the second transducer group 22. The image display function 534 displays the ultrasonic image of the second cross section via the output interface 4 (step S10).

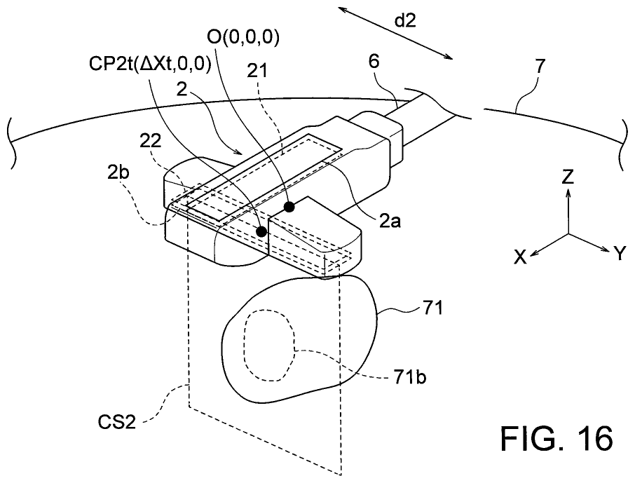

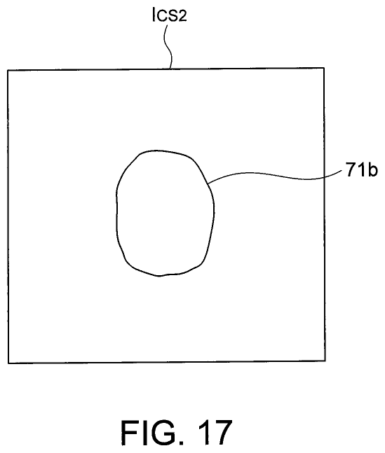

FIG. 16 is an explanatory diagram for explaining a driving process of the second transducer group 22 in an operation example of the ultrasonic diagnostic apparatus 1 according to the first embodiment. FIG. 17 is an explanatory diagram for explaining a step of displaying an ultrasonic image of a second cross section in the operation example of the ultrasonic diagnostic apparatus 1 according to the first embodiment. In the example illustrated in FIG. 16, the position CP2 of the second transducer group 22 is moved from the initial position (ΔX2, 0, 0) at the time of the inversion illustrated in FIG. 13 to the target position CP2t (ΔXt, 0, 0). The ultrasonic probe 2 drives the second transducer group 22 at the target position CP2t (ΔXt, 0, 0) to scan the second cross section CS2 including the observation target 71 with ultrasonic waves. The image generation function 533 generates an ultrasonic image I_{CS2} of the second cross section CS2 including a cross section 71b of the observation target 71 on the basis of a scan result of the second transducer group 22. As illustrated in FIG. 17, the image display function 534 displays, via the output interface 4, the ultrasonic image I_{CS2} of the second cross section CS2 generated by the image generation function 533.

On the other hand, in a case where the second transducer group 22 has not reached the target position (step S9: No), as illustrated in FIG. 8, the guide function 539 continues guiding the second transducer group 22 to the target position (step S8).

Note that, in the example described above, the ultrasonic diagnostic apparatus 1 first drives the first transducer group 21 to acquire an ultrasonic image of the first cross section, and then drives the second transducer group 22 to acquire an ultrasonic image of the second cross section. The ultrasonic diagnostic apparatus 1 is not limited to first driving the first transducer group 21, and may first drive the second transducer group 22 to acquire an ultrasonic image of the second cross section, and then drive the first transducer group 21 to acquire an ultrasonic image of the first cross section. In this case, the above-described processing of the ultrasonic diagnostic apparatus 1 may be applied by replacing the first transducer group 21 and the second transducer group 22 with each other.

Furthermore, in the example described above, the setting function 531 sets the second transducer group 22 as a transducer group to be driven according to the input operation after the ultrasonic probe 2 is inverted. The setting function 531 is not limited to setting the transducer group to be driven according to the input operation, and may automatically set the second transducer group 22 as a transducer group to be driven in response to detection of the inversion of the ultrasonic probe 2 by the position sensor 23 such as an acceleration sensor, for example.

Furthermore, in the above-described example, the position recording function 538 records the position (origin) of the first transducer group 21 and the target position of the second transducer group 22 when the ultrasonic image of the first cross section is generated in the storage circuitry 52 before the ultrasonic probe 2 is inverted. The position recording function 538 is not limited to the recording before the inversion of the ultrasonic probe 2, and may record the position (origin) of the first transducer group 21 and the target position of the second transducer group 22 in the storage circuitry 52 in response to detection of the start of the inversion of the ultrasonic probe 2 by the position sensor 23 such as an acceleration sensor. That is, the position recording function 538 may record the position (origin) of the first transducer group 21 and the target position of the second transducer group 22 in the storage circuitry 52 simultaneously with the inversion of the ultrasonic probe 2.

Furthermore, in a case where the ultrasonic image of the first cross section is continuously displayed even after the inversion of the ultrasonic probe 2, the designation of the position of the second cross section on the ultrasonic image of the first cross section (step S3) and the calculation of the target position of the second transducer group 22 (step S4) may be performed after the inversion of the ultrasonic probe 2 (for example, between step S7 and step S8 in FIG. 8). In this case, the target position of the second transducer group 22 is calculated as XYZ coordinates with the position of the first transducer group 21 when the ultrasonic image of the first cross section is generated as an origin.

Furthermore, the guide function 539 may guide an aligning operation of aligning the position CP2 of the second transducer group 22 with the origin O (0, 0, 0) before or in the course of guiding the movement of the second transducer group 22 to the target position CP2t. The guide of the alignment operation may be performed using at least one of the image and the sound.

Furthermore, the position recording function 538 may record the position CP1 of the first transducer group 21 when the ultrasonic image of the first cross section is generated as it is in the storage circuitry 52 without setting the position CP1 as the origin. Then, after the ultrasonic probe 2 is inverted, the guide function 539 may align the position CP2 of the second transducer group 22 with the recorded position CP1 of the first transducer group 21, and then guide the movement of the second transducer group 22 to the target position.

Furthermore, in the example described above, the position detection function 535 detects the positions of the first transducer group 21 and the second transducer group 22 based on the electric signal from the position sensor 23. The position detection function 535 is not limited to the position detection only, and may further detect angles of the first transducer group 21 and the second transducer group 22 in addition to the positions of the first transducer group 21 and the second transducer group 22. That is, the position detection function 535 may function as an angle detection unit. When the position sensor 23 is a sensor such as an acceleration sensor that can also be used for angle detection, the position detection function 535 can detect the angles of the transducer groups 21 and 22 together with the positions of the transducer groups 21 and 22.

The position detection function 535 may detect, for example, at least one of an angle around the X axis, an angle around the Y axis, and an angle around the Z axis as the angles of the first transducer group 21 and the second transducer group 22. The angle detected by the position detection function 535 may be used as follows, for example.

Before the ultrasonic probe 2 is inverted, the position detection function 535 detects the position of the first transducer group 21 when the ultrasonic image of the first cross section is generated and the angle of the first transducer group 21. The position recording function 538 records the angle of the first transducer group 21 detected when the ultrasonic image of the first cross section is generated in the storage circuitry 52 in association with the position of the first transducer group 21. At this time, the position recording function 538 may record the angle of the first transducer group 21 detected when the ultrasonic image of the first cross section is generated in the storage circuitry 52 as a reference of the angle (for example, the angle around the X axis is 0°, the angle around the Y axis is 0°, and the angle around the Z axis is 0°).

After the ultrasonic probe 2 is inverted, the position detecting function 535 detects the position of the second transducer group 22 and the angle of the second transducer group 22. The angle of the second transducer group 22 detected by the position detection function 535 may be an angle with respect to the reference of the angle recorded by the position recording function 538. The guide function 539 guides the angle adjustment of the second transducer group 22 using the angle of the second transducer group 22 detected by the position detection function 535 so that the second cross section intersecting the first cross section at an angle set by the default setting or the input operation can be scanned.

The guide function 539 may guide the angle adjustment of the second transducer group 22 using at least one of the image and the sound. The guide of the angle adjustment may be performed, for example, by displaying the angle of the second transducer group 22 on the screen, displaying the deviation of the angle of the second transducer group 22 from the set angle (target angle) on the screen, outputting a beep sound when the angle of the second transducer group 22 becomes the set angle, and the like. The guide of the angle adjustment may be performed together with the guide of the alignment operation described above. Alternatively, the guide of the angle adjustment may be performed at the target position of the second transducer group 22. Alternatively, the guide of the angle adjustment may be continuously performed after the inversion of the ultrasonic probe 2. By guiding the angle adjustment of the second transducer group 22, it is possible to easily observe the intersecting cross section at a desired angle.

As described above, in the first embodiment, the ultrasonic probe 2 includes the first transducer group 21 including the plurality of transducers 21a arranged along the first direction d1, and the second transducer group 22 arranged in an inverted direction with respect to the first transducer group 21 and including the plurality of transducers 22a arranged along the second direction d2 different from the first direction d1. The first transducer group 21 and the second transducer group 22 are switched and driven with respect to each other.

As a result, after the first transducer group 21 is driven to acquire the ultrasonic image of the first cross section, the ultrasonic probe 2 is inverted and the second transducer group 22 is driven, whereby the ultrasonic image of the second cross section in a direction different from the first cross section can be acquired. Therefore, it is possible to acquire ultrasonic images of cross sections in different directions without almost rotating the ultrasonic probe 2 about an axis orthogonal to the cable 6 and without replacing the ultrasonic probe 2. As a result, observation of the cross section of the subject can be facilitated.

Furthermore, in the first embodiment, the plurality of transducers 22a of the second transducer group 22 is arranged in the second direction d2 orthogonal to the first direction d1. Furthermore, the ultrasonic image of the second cross section is an ultrasonic image of a cross section orthogonal to the first cross section.

This makes it possible to easily acquire an ultrasonic image of an orthogonal cross section.

Furthermore, in the first embodiment, the plurality of transducers 22a of the second transducer group 22 can be arranged in the second direction d2 intersecting the first direction d1 at an angle other than 90°.

As a result, the degree of freedom in designing the ultrasonic probe 2 can be improved.

Furthermore, in the first embodiment, the plurality of transducers 21a of the first transducer group 21 is arranged along the cable 6.

As a result, by performing a simple operation of inverting the ultrasonic probe 2 about the axis along the cable 6 and then moving the ultrasonic probe 2 along the cable 6, it is possible to more easily acquire the ultrasonic images of the cross sections in the directions different from each other.

Furthermore, in the first embodiment, the scanning method of the first transducer group 21 is the same as the scanning method of the second transducer group 22.

As a result, in a case where the first transducer group 21 and the second transducer group 22 are driven by the same scanning method, it is possible to easily acquire ultrasonic images of cross sections in different directions.

Furthermore, in the first embodiment, the scanning method of the first transducer group 21 can be different from the scanning method of the second transducer group 22.

As a result, even in a case where the first transducer group 21 and the second transducer group 22 are driven by different scanning methods, it is possible to easily acquire ultrasonic images of cross sections in different directions.

Furthermore, in the first embodiment, the scanning method of the first transducer group 21 and the second transducer group 22 is any of a linear electronic scanning method, a convex electronic scanning method, and a sector electronic scanning method.

As a result, the degree of freedom in designing the ultrasonic probe 2 can be improved.

Furthermore, in the first embodiment, the setting function 531 sets one of the first transducer group 21 and the second transducer group 22 as a transducer group to be driven. Furthermore, the drive function 532 drives the transducer group set by the setting function 531. Furthermore, the image generation function 533 generates an ultrasonic image of the subject on the basis of the reflected wave signal received by the transducer group driven by the drive function 532.

As a result, since the transducer group set by the setting function 531 can be driven, switching of driving between the first transducer group 21 and the second transducer group 22 can be easily and appropriately performed.

Furthermore, in the first embodiment, the position detection function 535 detects the positions of the first transducer group 21 and the second transducer group 22. Furthermore, the position recording function 538 records the detected position of the first transducer group 21 in the storage circuitry 52 when the position of the first transducer group 21 when the ultrasonic image of the first cross section is generated by driving the first transducer group 21 and the position detection function 535 generates the ultrasonic image of the first cross section is detected. Furthermore, when the ultrasonic image of the second cross section is generated by driving the second transducer group 22, the guide function 539 uses the position of the first transducer group 21 recorded in the storage circuitry 52 as a reference of the position of the second transducer group 22 to guide the movement of the second transducer group 22 to the position where the ultrasonic image of the second cross section is acquired.

As a result, based on the position of the first transducer group 21 when the ultrasonic image of the first cross section is generated, the movement of the second transducer group 22 can be guided to the position where the ultrasonic image of the second cross section is acquired, so that the ultrasonic image of the second cross section can be easily acquired.

Furthermore, in the first embodiment, the image display function 534 displays the ultrasonic image of the first cross section. Furthermore, the cross-sectional position designation function 536 receives an operation of designating the position of the second cross section on the ultrasonic image of the first cross section displayed by the image display function 534. Furthermore, the guide function 539 guides the movement of the second transducer group 22 according to the position of the second cross section designated by the designation operation.

As a result, by guiding the movement of the second transducer group 22 according to the position of the second cross section designated on the ultrasonic image of the first cross section, it is possible to easily acquire the ultrasonic image of the second cross section desired by the operator.

Furthermore, in the first embodiment, the target position calculation function 537 calculates the target position of the second transducer group 22 corresponding to the position of the second cross section designated by the designation operation. Furthermore, the guide function 539 guides the movement of the second transducer group 22 to the position calculated by the target position calculation function 537, thereby guiding the movement of the second transducer group 22 according to the position of the second cross section.

As a result, since the second transducer group 22 can be appropriately guided according to the target position calculated by the target position calculation function 537, it is possible to easily and appropriately acquire the ultrasonic image of the second cross section desired by the operator.

Furthermore, in the first embodiment, the setting function 531 sets one of the first transducer group 21 and the second transducer group 22 as a transducer group to be driven according to an input operation of the operator.

As a result, since the transducer group desired by the operator can be driven, it is possible to acquire an ultrasonic image of a cross section desired by the operator.

Furthermore, in the first embodiment, the guide function 539 guides the movement of the second transducer group 22 using at least one of an image and a sound.

This makes it possible to easily and appropriately guide the movement of the second transducer group 22.

### Second Embodiment

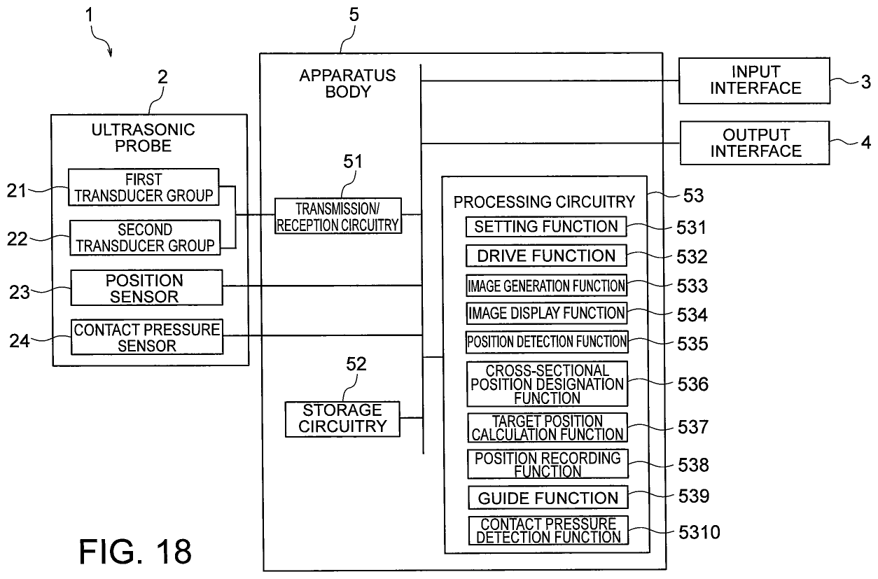

Next, a second embodiment in which a transducer group to be driven is set on the basis of a contact pressure will be described. FIG. 18 is a block diagram illustrating an example of a configuration of an ultrasonic diagnostic apparatus 1 according to the second embodiment. The setting function 531 in the first embodiment sets a transducer group to be driven according to an input operation of an operator. On the other hand, a setting function 531 in the second embodiment is configured to set a transducer group to be driven on the basis of a contact pressure.

Specifically, as illustrated in FIG. 18, the ultrasonic probe 2 in the second embodiment further includes a contact pressure sensor 24 in addition to the configuration of the first embodiment. Furthermore, a processing circuitry 53 in the second embodiment further includes a contact pressure detection function 5310 in addition to the configuration of the first embodiment. The contact pressure sensor 24 and the contact pressure detection function 5310 are examples of a contact pressure detector. The contact pressure sensor 24 is a sensor for detecting the contact pressure of the first transducer group 21 and the second transducer group 22. A specific mode of the contact pressure sensor 24 is not particularly limited, and may be, for example, a sensor that outputs an electric signal having a signal value according to the contact pressure to the processing circuitry 53. For example, the contact pressure sensor 24 may be a piezoelectric sensor including a piezoelectric element provided corresponding to the first transducer group 21 and a piezoelectric element provided corresponding to the second transducer group 22. The contact pressure detection function 5310 calculates (that is, detects) contact pressures of the first transducer group 21 and the second transducer group 22 based on the electric signal output from the contact pressure sensor 24.

The setting function 531 sets one of the first transducer group 21 and the second transducer group 22 as a transducer group to be driven on the basis of the contact pressures of the first transducer group 21 and the second transducer group 22 detected by the contact pressure detection function 5310. Specifically, the setting function 531 sets one of the first transducer group 21 and the second transducer group 22 having a large detected contact pressure as the transducer group to be driven.

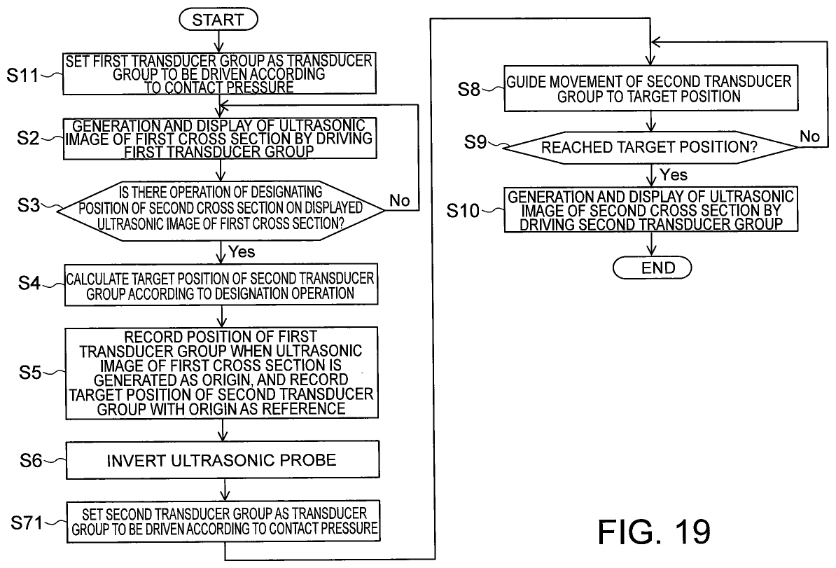

Next, an operation example of the ultrasonic diagnostic apparatus 1 according to the second embodiment will be described focusing on a difference from the first embodiment. FIG. 19 is a flowchart illustrating an operation example of the ultrasonic diagnostic apparatus 1 according to the second embodiment.

In the example illustrated in FIG. 19, the operator first brings the first acoustic surface 2a corresponding to the first transducer group 21 into contact with the internal surface 7 of the subject as illustrated in FIG. 9. As a result, the contact pressure detection function 5310 detects a contact pressure of the first transducer group 21 larger than a contact pressure of the second transducer group 22. At this time, the contact pressure of the second transducer group 22 may be 0. Then, as illustrated in FIG. 19, the setting function 531 sets the first transducer group 21 as a transducer group to be driven according to the contact pressure detected by the contact pressure detection function 5310 (step S11). Thereafter, the same series of steps (steps S2 to S6) as in the first embodiment are performed until the ultrasonic probe 2 is inverted (step S6).

By inverting the ultrasonic probe 2 in step S6, the operator brings the second acoustic surface 2b corresponding to the second transducer group 22 into contact with the inner surface 7 of the subject as illustrated in FIG. 13. As a result, the contact pressure detection function 5310 detects a contact pressure of the second transducer group 22 larger than a contact pressure of the first transducer group 21. At this time, the contact pressure of the first transducer group 21 may be 0. Then, as illustrated in FIG. 19, the setting function 531 sets the second transducer group 22 as a transducer group to be driven according to the contact pressure detected by the contact pressure detection function 5310 (step S71). Thereafter, the same series of steps as in the first embodiment (steps S8 to S10) are performed.

As described above, in the second embodiment, the setting function 531 sets the transducer group to be driven on the basis of the contact pressure detected by the contact pressure detection function 5310.

As a result, the operation of setting the transducer group to be driven can be omitted, so that the cross section of the subject can be more easily observed. Furthermore, the transducer group to be driven can be simply and appropriately set on the basis of the contact pressure.

Note that the term “processor” used in the above description means, for example, a central processing unit (CPU), a graphics processing unit (GPU), or a circuitry such as an application specific integrated circuitry (ASIC) or a programmable logic device (for example, a simple programmable logic device (SPLD), a complex programmable logic device (CPLD), and a field programmable gate array (FPGA)). The processor realizes the function by reading and executing the program stored in the storage circuitry. Note that instead of storing the program in the storage circuitry, the program may be directly incorporated in the circuitry of the processor. In this case, the processor realizes the function by reading and executing a program incorporated in the circuitry. Note that the processor is not limited to a case of being configured as a single processor circuitry, and a plurality of independent circuitries may be combined to be configured as one processor to realize the function. Moreover, the plurality of components in FIG. 1 may be integrated into one processor to implement the function.

According to at least one embodiment described above, observation of a cross section of a subject can be facilitated.

Although several embodiments have been described above, these embodiments have been presented only as examples, and are not intended to limit the scope of the invention. The novel devices and methods described herein can be implemented in a variety of other forms. Furthermore, various omissions, substitutions, and changes can be made to the forms of the apparatus and the method described in the present specification without departing from the gist of the invention. The appended claims and their equivalents are intended to include such forms and modifications as fall within the scope and spirit of the invention.

What is claimed is:

1. An ultrasonic probe comprising:
a first transducer group including a plurality of transducers arranged along a first direction; and
a second transducer group arranged in an inverted direction with respect to the first transducer group, the second transducer group including a plurality of transducers arranged along a second direction different from the first direction.

2. The ultrasonic probe according to claim 1, wherein the first transducer group and the second transducer group are driven while being switched with each other.

3. The ultrasonic probe according to claim 1, wherein the second direction is a direction orthogonal to the first direction.

4. The ultrasonic probe according to claim 1, wherein the second direction is a direction intersecting the first direction at an angle other than 900.

5. The ultrasonic probe according to claim 1, further comprising
a cable electrically connected to the first transducer group and the second transducer group,
wherein the plurality of transducers of the first transducer group or the plurality of transducers of the second transducer group are arranged along the cable.

6. The ultrasonic probe according to claim 1, wherein a scanning method of the first transducer group is a same as a scanning method of the second transducer group.

7. The ultrasonic probe according to claim 1, wherein a scanning method of the first transducer group is different from a scanning method of the second transducer group.

8. The ultrasonic probe according to claim 6, wherein a scanning method of the first transducer group and the second transducer group is any of a linear electronic scanning method, a convex electronic scanning method, and a sector electronic scanning method.

9. An ultrasonic diagnostic apparatus comprising:
an ultrasonic probe configured to transmit an ultrasonic wave to a subject and receive a reflected wave signal of the ultrasonic wave, the ultrasonic probe including a first transducer group including a plurality of transducers arranged along a first direction, and a second transducer group arranged in an inverted direction with respect to the first transducer group, the second transducer group including a plurality of transducers arranged along a second direction different from the first direction; and
a processing circuitry configured to set one of the first transducer group and the second transducer group as a transducer group to be driven,
drive the set transducer group, and
generate an ultrasonic image of the subject based on the reflected wave signal received by the driven transducer group.

10. The ultrasonic diagnostic apparatus according to claim 9, wherein the processing circuitry is further configured to
detect a position of the first transducer group and a position of the second transducer group,
when the ultrasonic image of a first cross section of the subject corresponding to the first transducer group is generated by driving of the first transducer group and the position of the first transducer group is detected when the ultrasonic image of the first cross section is generated, record the detected position of the first transducer group in a memory, and
when the ultrasonic image of a second cross section of the subject corresponding to the second transducer group is generated by driving of the second transducer group, guide movement of the second transducer group to a position where the ultrasonic image of the second cross section is acquired by using the position of the first transducer group recorded in the memory as a reference of the position of the second transducer group.

11. The ultrasonic diagnostic apparatus according to claim 10, wherein the ultrasonic image of the second cross section is the ultrasonic image of a cross section orthogonal to the first cross section.

12. The ultrasonic diagnostic apparatus according to claim 10, wherein the processing circuitry is further configured to
display the ultrasonic image of the first cross section,
receive a designation operation of a position of the second cross section on the displayed ultrasonic image of the first cross section, and
guide the movement of the second transducer group according to the position of the second cross section designated by the designation operation.

13. The ultrasonic diagnostic apparatus according to claim 12, wherein the processing circuitry is further configured to
calculate a target position of the second transducer group corresponding to the position of the second cross section designated by the designation operation, and
guide the movement of the second transducer group according to the position of the second cross section by guiding the movement of the second transducer group to the calculated target position.

14. The ultrasonic diagnostic apparatus according to claim 9, wherein the processing circuitry is further configured to set one of the first transducer group and the second transducer group as a transducer group to be driven according to an input operation of an operator.

15. The ultrasonic diagnostic apparatus according to claim 9, wherein the processing circuitry is further configured to
detect contact pressure of the first transducer group and contact pressure of the second transducer group, and
set one of the first transducer group and the second transducer group as a transducer group to be driven based on the detected contact pressure of the first transducer group and the detected contact pressure of the second transducer group.

16. The ultrasonic diagnostic apparatus according to claim 15, wherein the processing circuitry is configured to set one of the first transducer group and the second transducer group having the larger contact pressure as the transducer group to be driven.

17. The ultrasonic diagnostic apparatus according to claim 10, wherein the processing circuitry is configured to guide the movement of the second transducer group using at least one of an image and a sound.

18. The ultrasonic diagnostic apparatus according to claim 10, wherein the processing circuitry is configured to
detect an angle of the first transducer group together with a position of the first transducer group, and detect an angle of the second transducer group together with a position of the second transducer group,
record the angle of the first transducer group detected when the ultrasonic image of the first cross section is generated in the memory in association with the position of the first transducer group detected when the ultrasonic image of the first cross section is generated, and
guide angle adjustment of the second transducer group so that the second cross section intersecting the first cross section at a set angle can be scanned using the angle of the first transducer group recorded in the memory as a reference of the angle of the second transducer group.

19. The ultrasonic diagnostic apparatus according to claim 18, wherein the set angle is a default set angle.

20. The ultrasonic diagnostic apparatus according to claim 18, wherein the set angle is an angle set by an input operation.
